(19) United States
(12) Patent Application Publication
(10) Pub. No.: US 2008/0146672 A1
(43) Pub. Date: Jun. 19, 2008

(54) Topical Eutectic Anesthetic Composition for Oral or Dermal Tissue

(76) Inventors: Marcia Marye DENTON, Kerrville, TX (US); Jerry A. Rogers, Kerrville, TX (US)

Correspondence Address: KAMMER BROWNING PLLC, 7700 BROADWAY, SUITE 202, SAN ANTONIO, TX, 78209, US

(21) Appl. No.: 11/952,923

(22) Filed: Dec. 7, 2007

Related U.S. Application Data
(60) Provisional application No. 60/873,479, filed on Dec. 8, 2006.

(51) Int. Cl. A61K 31/16

(52) U.S. Cl. 514/626

(57) ABSTRACT

A pharmaceutical anesthetic composition for topical administration for dental, emergency and general medical use is described. The composition is a eutectic composition of anesthetic agents, sugar alcohol, and menthol for the purpose of numbing oral or dermal tissue. Methods of making and of using the composition are described.

### CROSS REFERENCE TO RELATED APPLICATIONS

This application claims the benefit under Title 35 United States Code § 119(e) of U.S. Provisional Application No. 60/873,479 filed Dec. 8, 2006, the full disclosure of which is incorporated herein by reference.

### BACKGROUND OF THE INVENTION

1. Field of the Invention

The present invention relates generally to anesthetic compositions for topical administration in the dental, emergency and general medical field. The present invention relates more specifically to a topical eutectic composition of anesthetic agents, sugar alcohol, and menthol for the purpose of numbing oral or dermal tissue.

2. Description of the Related Art

There are over 68 million individuals who delay or do not see a dentist on a regular basis. For most of these people, fear keeps them from making a dental appointment until they experience pain which outweighs their fear of going to the dentist. Research has shown that most of the people who do not see the dentist on a regular basis or only in a dental crisis have had a negative dental experience in the past. Many or these negative experiences occurred as young children, and many of these occurred because of painful injections or procedures. The negative consequences for injection and treatment pain is true for children in large part because the numbing injection causes pain prior to the eventually numbing that allows for treatments including sutures, dental carries, and scaling. This event becomes a traumatic event with consequences of adult avoidance behavior.

Absence of dental care can be very detrimental to a person's well-being and health. A dentist is able to determine a person's overall health by observing the oral cavity, checking for cancer or precancerous conditions, and assessing gum health. The dentist thus becomes a gateway to monitoring the systemic health of an individual. Preventive health is a large part of a dentist's responsibilities and is extremely important in reducing health costs and increasing longevity and quality of life. However, if a person does not go to a dentist, this gateway to preventative health is lost.

Currently, topical products which are available to dentists and other medical professionals provide only partial pain relief and do not numb sufficiently to eliminate the need for injections in some cases nor prevent an injection from being painful. Additionally, in many states, a dentist or doctor is required to personally administer such injections, including for a procedure called deep root scaling and planning which is normally performed by a dental hygienist. This can be a potentially painful or very uncomfortable procedure if done without an injection. Topical numbing products on the market do not always provide sufficient numbing for such procedures, many require an injection before the procedure can be performed or continued if the patient perceives too much pain. Because the injection is required to be administered by a dentist, the dentist must stop the procedure he/she is currently doing, wash his/her hands, re-mask, and attend to the dental hygienist's patient while his/her patient is waiting. It would be beneficial to have a topical product which could be applied by a dental hygienist and which would provide sustained deep numbing without the need of an injection for such procedures.

Although there are products on the market that are used for topical numbing in dentistry and medicine, they generally do not provide a sustained deep numbing effect. It would be beneficial to have a topical eutectic gel which provides superior numbing for an extended period of time for oral or dermal use.

### SUMMARY OF THE INVENTION

In fulfillment of the above described needs in the dental and/or medical health care field, the present invention provides a topical eutectic gel having a sustained deep numbing effect, thus providing pain free injections and the performance of other procedures with only a topical anesthetic which previously required painful injections.

The present invention is a unique combination of four types of substances: anesthetic agents from both the amide and amine families, sugar alcohols, and terpenes such as menthol that in combination provides a more potent effect than used alone or in a more limited combination. This novel combination can be used as a topical numbing agent prior to injections, sutures, mole removal, cauterizing lesions, lazering of lesions, setting of crowns and other procedures as well as a periodontal pocket numbing agent prior to deep root scaling and planing. In many cases it eliminates the need for an injection. Certain dental procedures such as regular fillings and root canals will continue to require injections, but the painfulness of the injection will be reduced and even eliminated through preparatory use of the present invention.

Due to the efficacy and potency of this product, there is great potential for its use in developing countries. Many of these countries do not have sufficient injectable and topical anesthetics to take care of minor dentistry and medical needs. With this product there is no need for refrigeration or special containers. It can simply be applied with an applicator to facilitate humane dental or medical treatment.

However, the greatest potential advantage of this product is its use for pediatric patients. Given the strong negative effect of a painful injection and how such traumatic early experiences frequently result in later avoidance of dental care, it is of the utmost importance that a product be made available that can eliminate or reduce such lasting negative effects. Improved dental experiences will yield increased willingness to seek dental care over a person's life span, resulting in improvements in preventative dental care and systemic wellness monitoring.

### BRIEF DESCRIPTION OF THE DRAWINGS

FIG. 1 is a detailed view, partially schematic, of a neuron synapse showing the pre-synaptic neuron and the post-synaptic neuron.

FIG. 2A is a detailed view of a voltage gated channel in a closed condition.

FIG. 2B is a diagram of a voltage gated channel in an open condition.

FIG. 2C is a diagram of a ligand gated channel in its closed condition.

FIG. 2D is a diagram of a ligand gated channel in its open condition.

FIG. 3 is a schematic overview of the de-polarizing and re-polarizing cycle that opens and closes the voltage gated channels.

FIG. 4 is a membrane potential graph showing the various regions of the voltage graph associated with stages in the de-polarizing and re-polarizing cycle.

FIG. 5A is a schematic view of paracellular transport as one mechanism for fluid movement through an epithelial cell layer.

FIG. 5B is a diagram showing the manner in which menthol acts on the TRPM8 receptor to depolarize the nerve and maintain depolarization.

FIG. 6 illustrates the structural formula for lidocaine.

FIG. 7 illustrates the structural formula for prilocalne.

FIG. 8 illustrates the structural formula for tetracaine.

FIG. 9 illustrates the structural formula for mannitol.

FIG. 10 illustrates the structural formula for menthol.

### DETAILED DESCRIPTION OF THE PREFERRED EMBODIMENTS

### 1. Background Regarding Nerve Synapse Impulse Mechanism

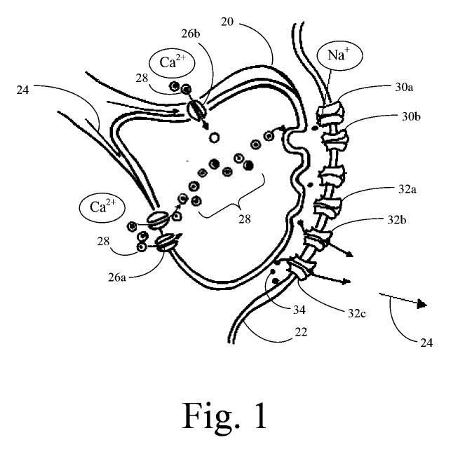

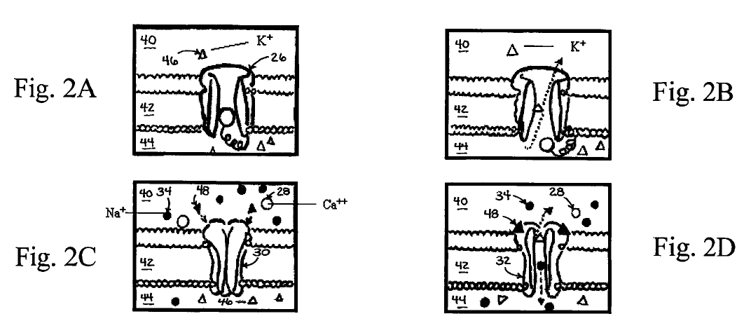

FIG. 1 provides a detailed view, partially schematic, of a neuron synapse showing the pre-synaptic neuron 20 and the post-synaptic neuron 22. Nerve impulse 24 flows through this synapse according to the mechanism generally described in FIG. 1. Voltage gated ion channels 26a and 26b (as examples) are positioned in the pre-synaptic neuron 20 synaptic end bulb. Calcium ions (Ca²⁺) 28 are allowed to move through these voltage gated channels 26 according to the potential across the membrane (described in more detail below with respect to FIGS. 2A & 2B). It is the calcium ions 28 that are therefore positioned within the synaptic end bulb 20 to carry neurotransmitters to a point where they may cross the synaptic cleft to activate the ligand gated channels 30a, 30b, 32a, 32b, and 32c (as examples). Ligand gated channels 30a and 30b as shown in FIG. 1 are closed and therefore do not permit the transfer of the sodium ions (Na⁺) 34 through the channels. Ligand gated channels 32a, 32b, and 32c (as examples) have been opened by the reception of a neurotransmitter or other ligand to the receptor site on the ligand gated channel. Once the neurotransmitters are received into the receptor site the ligand gated channel opens (as described in more detail below) and allows for the passage of the sodium ions 34. This post-synaptic potential that is established by the movement of the positively charged sodium ions through the ligand gated channels continues the nerve impulse 24 flow through the nerve synapse.

FIGS. 2A through 2B provide additional detail regarding the function of the gated ion channels described above in conjunction with the nerve synapse in FIG. 1. In each of the views shown in FIGS. 2A through 2D, the gated ion channels extend between the extra cellular fluid 40 through the plasma membrane 42 into the cytosol 44 of the cell. FIG. 2A shows a detailed view of a voltage gated channel in a closed condition, while FIG. 2B shows the same voltage gated channel in an open condition. A change in the membrane potential opens the channel. The view in FIG. 2A may be typical of a voltage of −70 mV whereas the open channel condition shown in FIG. 2B would be present with a voltage of −50 mV. The open condition of the channel allows potassium ions (K⁺) 46 (as an example) to flow through the channel 26 as shown.

FIGS. 2C and 2D represent a ligand gated channel in its closed condition (FIG. 2C) and in its open condition (FIG. 2D). In this case, a chemical stimulus serves to open the channel as can be seen by the introduction of ligand molecule 48 into the channel 32 in a manner that stimulates its opening. In the example provided, sodium ions and potassium ions are allowed to move as indicated through the open ligand gated channel.

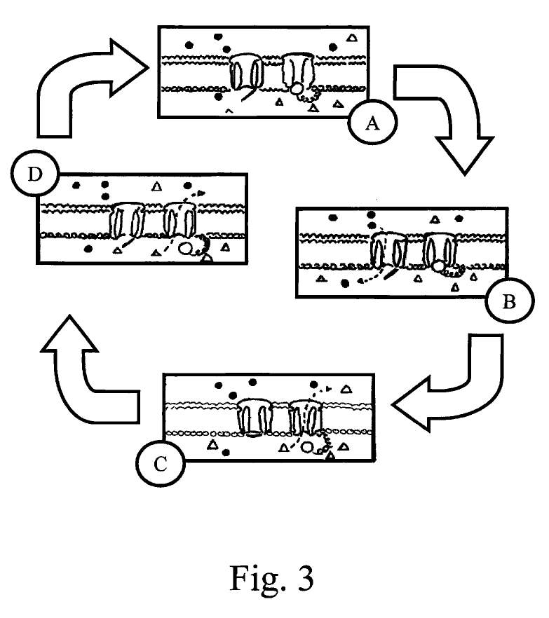

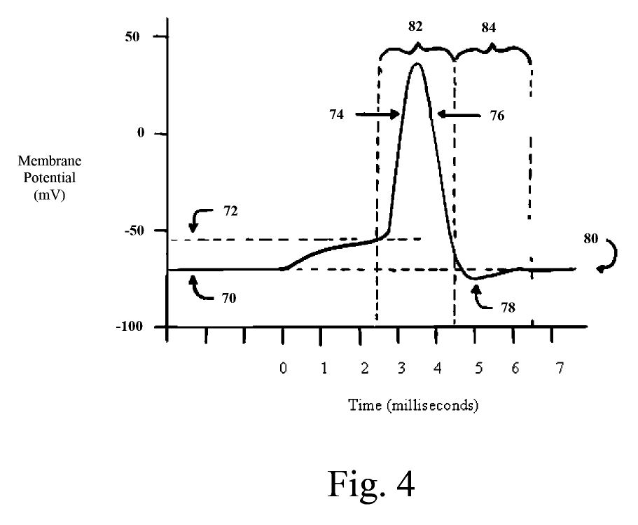

Reference is now made to FIG. 3 for a brief overview of the depolarizing and repolarizing cycle that opens and closes the voltage gated channels in a manner appropriate for the transfer of sodium and potassium ions across the plasma membrane. Reference is made in each stage of this cycle to the membrane potential graph shown in FIG. 4 wherein the various regions of the voltage graph are associated with stages in the depolarizing and repolarizing cycle.

At the first resting stage (A) shown in FIG. 3, the membrane potential is at approximately −70 mV (identified as resting potential region 70 in FIG. 4). Depolarization (B) represents a change from the −70 mV to a value of approximately +30 mV in the depolarizing phase. The depolarization to threshold potential 72 (FIG. 4) opens the sodium ion (Na⁺) channel activation gates. Sodium ions inflow further depolarizing the membrane opening additional sodium ion channel activation gates. This polarization 74 occurs over the action potential region 82 of the graph shown in FIG. 4.

Repolarization 76 (FIG. 4) then occurs as the voltage once again reverses from +30 mV back to −70 mV. In the repolarizing phase (C) the sodium ion channel inactivation gates close and the potassium channels open. An outflow of potassium ions causes repolarization to occur across the plasma membrane. As repolarization continues (D), the potassium ion outflow restores the resting membrane potential (−70 mV) 80 (FIG. 4). The sodium ion channel inactivation gates open as a result. This returns the gates to a resting state when the potassium ion gates close. This stage of the cycle is reflected in the refractory period portion 84 of the graph shown in FIG. 4.

Local anesthetics in certain neurotoxins are known to prevent the opening of voltage gated sodium channels. As a result, nerve impulses cannot pass the anesthetized region. Examples of such local anesthetics known to carry out this functionality are Novocain and Lidocaine. The process described above involves the propagation of an action potential. An action potential spreads (propagates) over the surface of the axon membrane as sodium ions flow into the cell. During depolarization, the voltage of the adjacent areas is affected and their voltage gated sodium ion channels open. This action self-propagates along the membrane due to the change in potential. A traveling action potential results in a nerve impulse.

There are two types of conduction that may occur according to the mechanisms described. These include continuous conduction versus saltatory conduction. Continuous conduction (unmyelinated fibers) comprises step-by-step depolarization of each portion of the length of the axolemma. The saltatory conduction process carries out depolarization only at nodes of Ranvier where there is a high density of voltage gated ion channels and current is carried by ion flows through the extra cellular fluid from node to node.

Local anesthetics provoke reversible blockade of nerves by interaction with sodium channels in membranes of nerves. The uncharged molecular configuration of the local anesthetic penetrates the membrane from the outside and the charged configuration then interacts with the sodium channel from the inside. The anesthetic inhibits voltage-gated sodium channels via the binding of drug molecules to these channels. Nerve impulses cannot pass the anesthetized region. The binding of drug molecules to these channels depends on their conformation, with the drugs generally having a higher affinity for the open and inactivated channel states that are induced by membrane depolarization. The potency of a local anesthetic is determined mainly by lipid solubility, the time of onset by the acid-ionization constant (pK(a)) of the substance and the duration of action by protein binding. Local anesthetic molecules consist of a hydrophilic tertiary amine and a lopophilic aromatic system combined by an ester or amide linkage. Local anesthetics may therefore be classified as aminoester or aminoamide compounds.

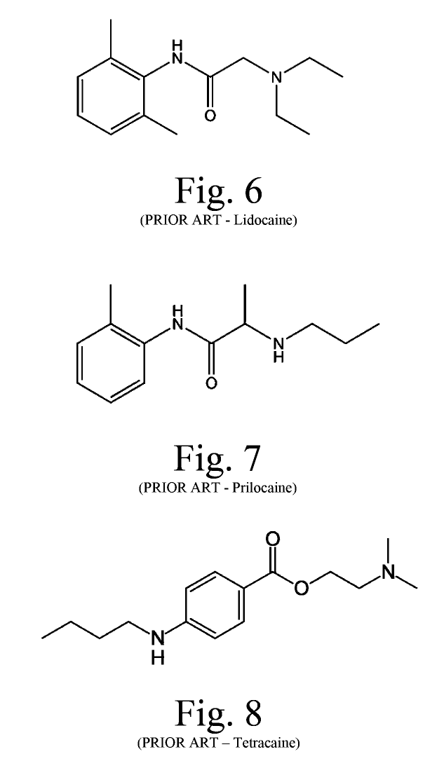

Research indicates that lidocaine (FIG. 6) and prilocalne (FIG. 7) are amino amide type anesthetics that preferentially inhibit chemotaxis, whereas tetracaine (FIG. 8) is an amino ester type anesthetic which inhibits superoxide anion (SOA) production induced by the bacterial tripeptide fMet-Leu-Phe (fMLP)1, one of the most powerful leukocyte chemoattractants. Lidocaine is the most widely used local anesthetic agent because of inherent potency, rapid onset, tissue penetration and effectiveness. Prilocalne is a local anesthetic of the amide class having an intermediate duration of action and is longer acting than lidocaine. Lidocaine and prilocalne are combined in a topical formulation for use on intact skin for local analgesia. An example is EMLA cream, which provides dermal analgesia by the release of lidocaine and prilocalne into the epidermal and dermal layers of the skin and accumulation of drug near dermal pain receptors. Tetracaine is an ester-type local anesthetic with an intermediate to long duration of action. These anesthetics cause a numbing effect by blocking the Ca²⁺ and Na⁺ ion channels, thus preventing a repolarization of the nerve and causing a temporal pain nerve blockage, i.e., numbing. This prevents additional action potentials and stops the sensations of pain for about twenty to thirty minutes.

### 2. Preferred Embodiment

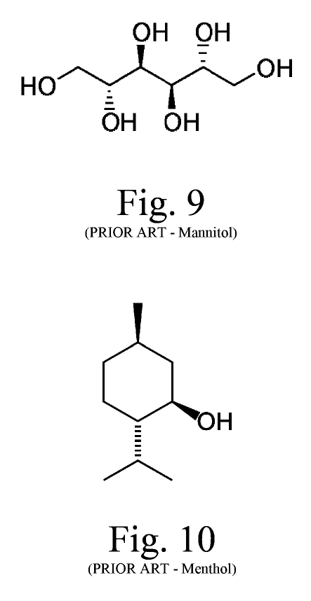

The present invention is a formulation of a eutectic anesthetic gel which combines lidocaine, prilocalne, and tetracaine with a sugar alcohol such as mannitol (FIG. 9) (a sorbitol stereoisomer) and the excipient menthol (FIG. 10) to provide a topical gel capable of numbing tissue within the oral cavity as well as topically for dermal applications. The product contains sufficient binders and inactive ingredients to form a eutectic mixture, capable of changing phase from liquid to gel/solid at body temperature and staying in place within the oral cavity. In addition, the formulation may at times use emulsion agents, thus becoming a microemulsion.

Local anesthetics block both the initiation and conduction of nerve impulses by decreasing the neuronal membrane's permeability to sodium ions. This reversibly stabilizes the membrane and inhibits depolarization, resulting in the failure of a propagated action potential and subsequent conduction blockade. A sugar alcohol, when added to the lidocaine, prilocalne, and tetracaine, potentiates the numbing effects by disrupting the nerve covering or sheath, thus enhancing the anesthetic effect. As the sugar alcohol level rises, intracellular myoinositol (important in cell membrane potential maintenance) level falls, which depletes Na+/K+-ATPase, (electrogenic transmembrane ATPase). This enzyme is responsible for the propagation of impulses along nerves, and the maintenance of proper conduction velocity.

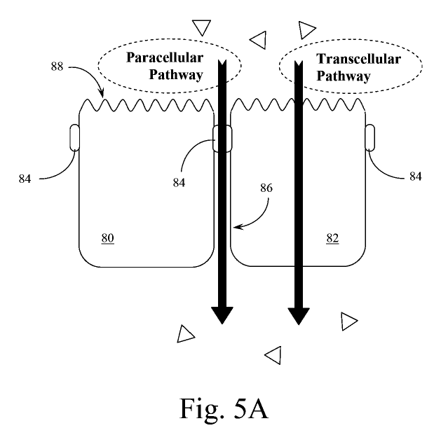

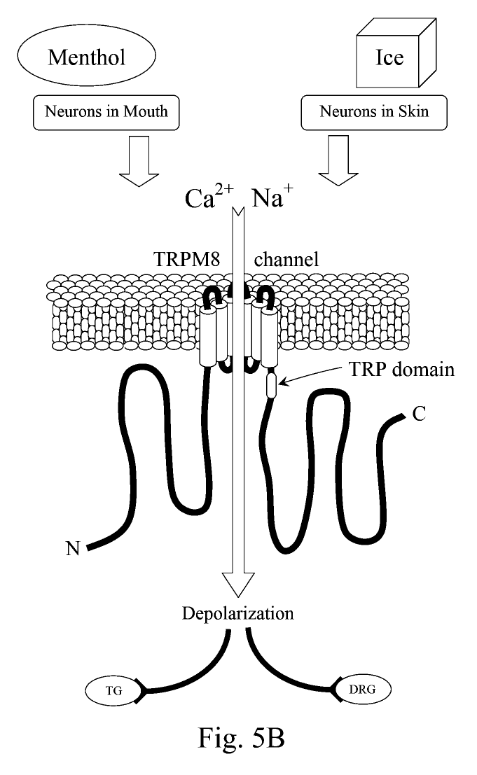

FIG. 5A provides a schematic view of paracellular transport as one mechanism for fluid movement through an epithelial cell layer. The figure contrasts paracellular transport with transcellular transport. Cells 80 & 82 of the epithelial layer (as an example) form what are referred to as tight junctions 84 and lateral space 86. Compounds may move across the cell layer from the apical surface 88 to the basolateral surface as shown. The mannitol is a hyperosmotic which actually causes a paracellular movement of fluid, i.e., the mannitol draws out the cellular fluid and shrinks the cells surrounding the nerve, thus allowing the anesthetic to move between and around the cells. This paracellular transport disrupts the perineural barrier of the nerve membrane and allows greater access of the anesthetics and menthol to the nerve. Thus mannitol also serves as a cell penetration enhancer, exposing the neuron by a breakdown of the myelin sheath, thus providing greater access to the nerve membrane by the anesthetics. The emulsion has a basic pH, non-ionized form which allows the anesthetic to readily cross the nerve membrane and helps extend the local analgesia. A more acidic substance prevents penetration, rendering the substance ineffective.

The menthol acts on the TRPM8 receptor (FIG. 5B) to depolarize the nerve and maintain depolarization so that the nerve impulses are blocked. TRPM8 is a nonselective cation channel, activated by cold temperatures, voltage, and menthol. The menthol acts as a TRPM8 ion channel agonist, binding to the receptor and triggering a response in the cell. It mimics the action of an endogenous ligand (such as a neurotransmitter) that binds to that same receptor. Menthol depolarizes the trigeminal and the dorsal root ganglia nerves, maintains depolarization, and has an analgesic effect on the brain and spinal cord. Topical anesthetics have an affinity for the open and inactivated state caused by depolarization, thus enhancing the effect of the numbing agents. This amplified effect combined with the anesthetic blocking the movement of Ca++ and Na+ results in a profoundly blocked nerve impulse.

The present invention is formulated in a series of steps requiring time for solutions and mixtures to dissolve. In the preferred embodiment, prilocalne, tetracaine, and lidocaine are mixed with the excipients mannitol and menthol. A minimum of three “caine” anesthetic substances are to be used but not necessarily the ones previously mentioned. Percents of each anesthetic range from 0.5% to 50% and can be adjusted in any ratio suitable for use. The combination of anesthetics is combined with any sugar alcohol (such as mannitol) in a percentage from 0.05% to 15%.

Using various solvents that will be evaporated, prilocalne is extracted from its HCl salt to a free base. Polysorbate 80 (emulsifier, surfactant) and propylene glycol (humectant, emulsifier, stabilizer) are used at various stages of the process to form a smooth, eutectic gel to which food coloring and flavoring may be added. The propylene glycol also enhances the penetration of agents in the emulsion into the tissue where it is applied, thus enhancing the time of onset and duration of the anesthetics. Terpene flavorings such as menthol (as peppermint oil) in a percentage from 0.05% to 15% and propylene glycol are also added and provide significant enhancement of the permeation of the cell membrane by the topical anesthetics.

The resulting compound may be applied in the dental environment with an endodontic or blunt dental needle within the periodontal cavity or applied by blunt or cotton applicator to the gum and pallet area.

Although the present invention has been described in terms of the foregoing preferred embodiments, this description has been provided by way of explanation only, and is not intended to be construed as a limitation of the invention. Those skilled in the art will recognize modifications of the present invention that might accommodate specific clinical requirements. Such modifications do not necessarily depart from the spirit and scope of the invention.

We claim:

1. A pharmaceutical composition for topical administration comprising: a therapeutically safe and effective amount of lidocaine or of a pharmaceutically acceptable salt thereof, a therapeutically safe and effective amount of prilocalne or of a pharmaceutically acceptable salt thereof, a therapeutically safe and effective amount of tetracaine or of a pharmaceutically acceptable salt thereof, a therapeutically safe and effective amount of mannitol or of a pharmaceutically acceptable sugar alcohol; and a therapeutically safe and effective amount of menthol or of a pharmaceutically acceptable terpene.

2. A method of use of a combination comprising lidocaine, prilocalne and tetracaine, any of which being as such or as a pharmaceutically acceptable salt, mannitol or a pharmaceutically acceptable sugar alcohol, and menthol or a pharmaceutically acceptable terpene for the preparation of a topical anesthetic pharmaceutical composition; the method comprising: providing a combination of lidocaine, prilocalne and tetracaine themselves or as pharmaceutically acceptable salts thereof, and mannitol or a pharmaceutically acceptable sugar alcohol; and menthol or a pharmaceutically acceptable terpene; and, administering the combination topically.
